(19) United States
(12) Patent Application Publication
(10) Pub. No.: US 2025/0065106 A1
(43) Pub. Date: Feb. 27, 2025

(54) DRIVELINE SYSTEM AND METHOD FOR AN IMPLANTABLE MEDICAL DEVICE

(71) Applicant: CH Biomedical, Inc., Suzhou (CN)

(72) Inventors: Jinjing Miao, Suzhou (CN); Xiao Ma, Suzhou (CN); Xuedan Tan, Suzhou (CN)

(21) Appl. No.: 18/453,008

(22) Filed: Aug. 21, 2023

(51) Int. Cl. A61M 60/873; A61M 60/508; A61M 60/585; H04L 27/18

(52) U.S. Cl. CPC A61M 60/873; A61M 60/508; A61M 60/585; H04L 27/18; A61M 2205/3538

(57) ABSTRACT

A driveline and driveline system for an active implantable device provides for communication over the power conductors in the driveline. The driveline includes four conductors, two for the positive power connection and two for the negative power connection. Communication in the driveline is performed using Power Line Communication (PLC) technology on the four conductors, superimposing a communication signal on the conductors such that the four conductors provide both power and communication capability. The four conductors provide redundant paths for both power and communication, so the failure of one of the conductors in the driveline does not affect operation of the driveline. The four conductor driveline cable and system provides a more robust driveline cable that does not fail with the failure of one of the conductors without increasing the diameter of the driveline, which would increase the risk of infection where the driveline passes through the skin.

### BACKGROUND

### 1. Technical Field

This invention generally relates to implantable medical devices, and more specifically relates to a driveline system and method for an implantable medical device.

### 2. Background Art

Implantable medical devices are implanted into a human body and help to prolong life and improve the patient's quality of life. Some implantable devices, such as pacemakers, have batteries that last for several years before they need to be replaced, and therefore have no need to be connected to a power supply external to the body. Some implantable devices are high-power active implantable devices, which consume sufficient power that a connection to an external power source is needed. This requires a cable commonly known as a driveline to be connected to the implantable device, then to pass through part of the patient's skin to an external device that includes or is connected to a power source. Examples of known high-power active implantable devices include: a left ventricular assist system (LVAS) device; a right ventricular assist device (RVAD); a bi-ventricular assist device (BiVAD); a percutaneous ventricular assist devices (pVAD); a mechanical circulatory system (MCS); and a total artificial heart (TAH).

### BRIEF SUMMARY

A driveline and driveline system for an active implantable device provides for communication over the power conductors in the driveline. The driveline includes four conductors, two for the positive power connection and two for the negative power connection. Communication in the driveline is performed using Power Line Communication (PLC) technology on the four conductors, superimposing a communication signal on the conductors such that the four conductors provide both power and communication capability. The four conductors provide redundant paths for both power and communication, so the failure of one of the conductors in the driveline does not affect operation of the driveline. The four conductor driveline cable and system provides a more robust driveline cable that does not fail with the failure of one of the conductors without increasing the diameter of the driveline, which would increase the risk of infection where the driveline passes through the skin.

The foregoing and other features and advantages of the invention will be apparent from the following more particular description of preferred embodiments of the invention, as illustrated in the accompanying drawings.

### BRIEF DESCRIPTION OF THE SEVERAL VIEWS OF THE DRAWING(S)

The preferred embodiments of the present invention will hereinafter be described in conjunction with the appended drawings, where like designations denote like elements, and:

FIG. 1 is a block diagram showing a prior art system that includes an implanted device with a driveline that passes through a patient's skin to an external device;

FIG. 2 is a cross-sectional view of a first prior art driveline;

FIG. 3 is a cross-sectional view of a second prior art driveline;

FIG. 4 is a block diagram of a sample system showing an implanted device that is coupled to an external controller, which is coupled to a power source;

FIG. 5 is a cross-sectional view of a prior art driveline that includes only two conductors;

FIG. 6 is a cross-sectional view of a driveline in accordance with the preferred embodiment that has four conductors;

FIG. 7 is a block diagram showing a driveline system in accordance with a preferred embodiment that includes an interconnection between first and second of the four conductors in the driveline shown in FIG. 6 and showing interconnection between the third and fourth conductors in the driveline;

FIG. 8 is a block diagram of a BPSK modulator;

FIG. 9 is a block diagram of a BPSK demodulator;

FIG. 10 is a block diagram of a system that is one possible implementation for the driveline system 700 in FIG. 7;

FIG. 11 is a block diagram of one possible implementation for the driveline communication transceivers shown in FIGS. 7 and 10;

FIG. 12 is a block diagram of one possible implementation for the driveline system in FIG. 7 using the SIG100 driveline communication transceiver;

FIG. 13 is a flow diagram of a method for initializing in the implanted device communication over the power conductors using the SIG100 driveline communication transceiver;

FIGS. 14-15 show a flow diagram of a method for initializing in the controller communication over the power conductors using the SIG100 driveline communication transceiver;

FIG. 16 is a block diagram that shows interconnecting the conductors in the driveline on a printed circuit board for the driveline communication transceiver;

FIG. 17 is a block diagram that shows interconnecting the conductors in the driveline in a first connector;

FIG. 18 is a block diagram that shows interconnecting the conductors in the driveline in a second connector;

FIG. 19 is a block diagram that shows interconnecting the conductors in the driveline in the driveline itself; and

FIG. 20 is a flow diagram of a method in accordance with the preferred embodiment.

### DETAILED DESCRIPTION

A driveline and driveline system for an active implantable device provides for communication over the power conductors in the driveline. The driveline includes four conductors, two for the positive power connection and two for the negative power connection. Communication in the driveline is performed using Power Line Communication (PLC) technology on the four conductors, superimposing a communication signal on the conductors such that the four conductors provide both power and communication capability. The four conductors provide redundant paths for both power and communication, so the failure of one of the conductors in the driveline does not affect operation of the driveline. The four conductor driveline cable and system provides a more robust driveline cable that does not fail with the failure of one of the conductors without increasing the diameter of the driveline, which would increase the risk of infection where the driveline passes through the skin.

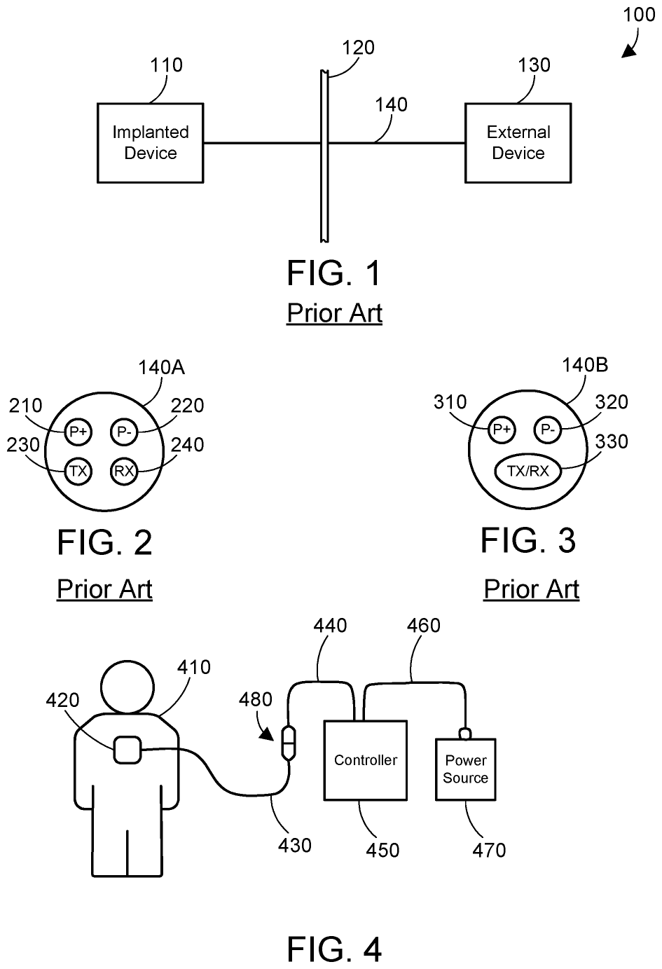

Referring to FIG. 1, a prior art system 100 includes an implanted device 110 that is implanted inside a patient's body, with a driveline 140 connected to the implanted device 110 that passes through the patient's skin 120 and connects to an external device 130. The external device 130 provides both power and communication to the implanted device 110 via the driveline 140. Two examples of prior art drivelines 140 are shown in FIGS. 2 and 3 as drivelines 140A and 140B, respectively. Referring to FIG. 2, prior art driveline 140A includes four conductors 210, 220, 230 and 240. The conductor 210 provides a positive polarity power connection while the conductor 220 provides a negative polarity power connection. The conductor 230 provides a transmit signal TX from the external device 130 to the implanted device 110, while the conductor 240 provides a receive signal RX from the implanted device 110 to the external device 130. Note the TX conductor 230 will be an RX input to the implanted device 110, and the RX conductor 240 will be a TX output from the implanted device 110. Most known drivelines for active medical devices include two or more pairs of conductors. For example, known drivelines for the HeartMate II and HeartMate 3 LVADs include six conductors. The Heartmate II and HeartMate 3 LVAD devices are marketed by Abbot. Heartmate II and HeartMate 3 are registered trademarks of TC1 LLC, a California limited liability company.

Referring to FIG. 3, an alternative prior art configuration provides a first pair of conductors 310 and 320 for power, and a single conductor 330 over which messages are both transmitted and received.

FIG. 4 shows a sample configuration showing an implanted device 420 inside the body 410 of a patient. The driveline in FIG. 4 is made up of the cables 430 and 440 that interconnect the implanted device 420 and the controller 450. These two cables 430 and 440 are connected together via mating connectors 480, and once connected together, make up the driveline as disclosed and claimed herein. Of course, a driveline as disclosed and claimed herein need not necessarily include connectors between two separate parts of the driveline. Instead the driveline could be a single cable that is connected to a connector on the controller 450 and includes either a hard-wired connection on the implanted device 420 or a waterproof connector on the implanted device 420. The controller 450 is connected via cable 460 to a power source 470. The power source 470 can include any suitable source of alternating current (AC) or direct current (DC) power, including a plug to a wall receptacle, a battery, a power supply that receives power from a wall receptacle or a battery, or any suitable combination of these powers sources, such as a rechargeable battery that can be charged via an AC outlet. The controller 450 can also include any suitable power source, such as a rechargeable battery, that can power the controller independently from the power source 470 when the controller 450 is unplugged from the power source 470. In this configuration, the controller 450 can function without the external power source 470 for some period of time using its own internal rechargeable battery (or batteries), which can then be recharged automatically once the controller 450 is reconnected to the power source 470. While the power source 470 is shown in FIG. 4 to be separate from the controller 450, the power source 470 could also be incorporated internal to the controller 450.

Infections caused by driveline 140 at the site where the driveline 140 passes through the skin 120 is a common adverse event in the clinical application of a high-power active implantable medical device, often causing serious complications and re-admission of the patient. Efforts are being made to optimize the driveline characteristics and implant technology to reduce driveline infections. Since a smaller outer diameter of driveline 140 is considered to be related to a lower probability of a driveline infection, minimizing the outer diameter of driveline 140 is one of the most direct and effective measures to reduce the probability of driveline infections.

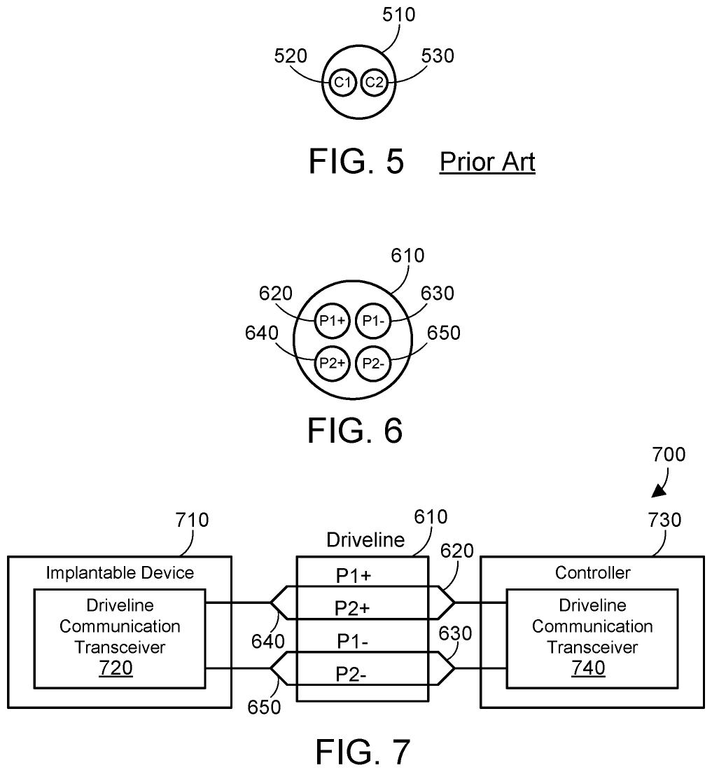

U.S. Pat. No. 9,308,305 issued on Apr. 12, 2016 to Chen et al. and assigned to the applicant of this patent application discloses in FIG. 7 and in col. 5 lines 54-67 a driveline that includes only two conductors for both power and communication. A sample cross-sectional view of a prior art two-conductor driveline is shown in FIG. 5. Both power and data are carried on the two conductors 520 and 530, eliminating the need for any additional conductors for communication. The driveline 510 in FIG. 5 is connected to the implantable device on one end and to the controller on the other end.

The prior art driveline 510 in FIG. 5 eliminates the need for a separate pair of conductors 230 and 240 shown in FIG. 2 for transmit/receive of digital messages, and even eliminates the single conductor 330 shown in FIG. 3. By eliminating conductors dedicated to transmission and reception of messages in the driveline, the diameter of the driveline can be reduced, which reduces the likelihood of driveline infection at the site where the driveline passes through the skin.

Over time, a driveline to an implanted device can bend and rub, which can create a failure in one of the conductors. In addition, a failure can occur, for example, from the chronic effects of moisture in the patient's body which can corrode contacts and wire and thereby reduce the impedance of a conductor to the point that failure of the conductor occurs. If a failure occurs in any of the four conductors shown in prior art driveline 140A in FIG. 2, either power or communication is lost. Likewise, if a failure occurs in any of the three conductors shown in the prior art driveline 140B shown in FIG. 3, either power or communication is lost. If a failure occurs in one of the two conductors in the prior art driveline 510 shown in FIG. 5, both power and communication is lost. With a failure in one conductor in any of these three prior art drivelines in FIGS. 2, 3 and 5, emergency measures must typically be taken to replace the implantable device and/or driveline very quickly to save the patient.

A driveline and driveline system in accordance with the preferred embodiment includes a driveline with four conductors as shown in FIGS. 6, 7 and 16-19. The four conductors provide redundant power connections, which means both power and communication can be maintained when one or even two of the conductors fail.

Providing a driveline that provides both redundant power and redundant communication on the same conductors would not have been obvious to one of ordinary skill in the art. Known drivelines create redundancy by increasing the number of conductors. For example, the cable for the HeartMate 3 LVAD includes six conductors, two positive polarity power conductors, two negative polarity power conductors, and two communication conductors, where each communication conductor can communicate bidirectionally. The HeartMate 3 LVAD thus provides redundant physical power connections and redundant physical data connections, thereby requiring six physical conductors in the driveline. But a driveline with six conductors will have a greater diameter than a driveline with four. The physical redundancy in the driveline for the HeartMate 3 LVAD thus comes at the expense of a higher likelihood of infection at the driveline site. Providing redundant power and communication capabilities using only the four conductors as shown in FIGS. 6 and 7 allows greatly enhanced reliability by providing redundancy using the same number of conductors as the prior art driveline shown in FIG. 2 without increasing the diameter of the driveline.

Referring to FIG. 6, a driveline 610 includes four conductors 620, 630, 640 and 650. The driveline system 700 in FIG. 7 includes the driveline 610 shown in FIG. 6 connected to a driveline communication transceiver 740 in the controller 730 on one end and connected to a driveline communication transceiver 720 in the implantable device 710 on the other end. In the most preferred implementation, two of the four conductors provide power in a first polarity and the remaining two conductors provide power in a second polarity. FIG. 7 shows both conductors 620 and 640 are connected to a positive polarity power source in the controller 730, as denoted by the P1+ and P2+ designations for conductors 620 and 640, respectively, while both conductors 630 and 650 are connected to a negative polarity power source in the controller 730, as denoted by the P1− and P2− designations for conductors 630 and 650, respectively. By providing redundant connections for both polarities, the failure of any one conductor will not affect the operation of the driveline system. In fact, one of the two conductors 620 and 640 could fail and one of the two conductors 630 and 650 could also fail without affecting the operation of the driveline system.

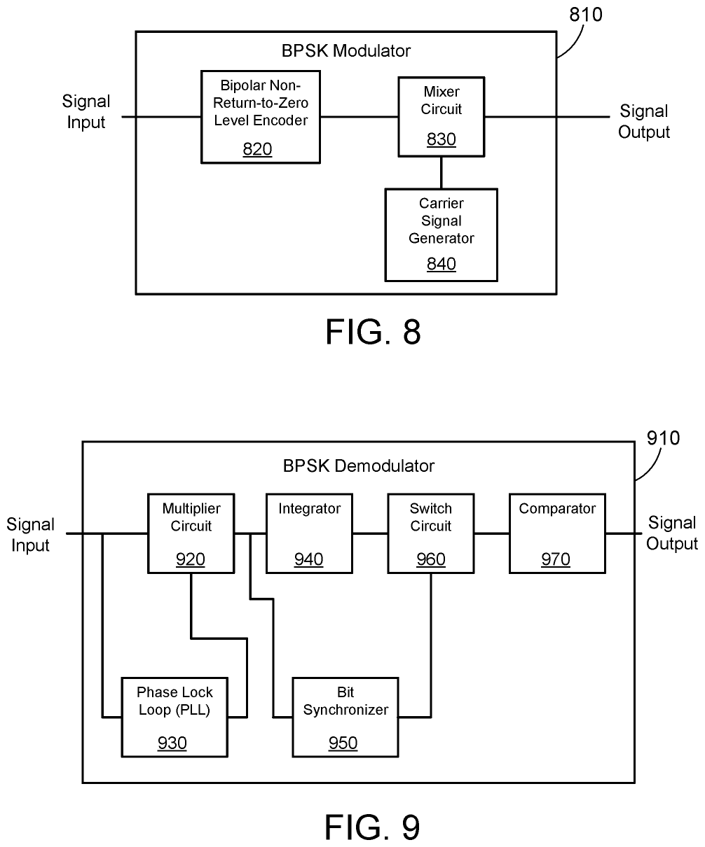

The driveline communication transceivers 720 and 740 in FIG. 7 each preferably include a BPSK modulator such as 810 shown in FIG. 8 and a BPSK demodulator such as 910 shown in FIG. 9. The driveline communication transceivers 720 and 740 thus allow communicating digital messages on the same two conductors that are used to provide power from the controller 730 to the to the implantable device 710.

The driveline of the preferred embodiment eliminates the need for a separate pair of conductors 230 and 240 shown in FIG. 2 for transmit/receive of digital messages, eliminates the single conductor 330 shown in FIG. 3, and provides redundancy in case of failure that is not present in prior art driveline 510 in FIG. 5. The transmission of digital messages over the same conductors used to provide power may be done using Power Line Communication (PLC) technology.

Power Line Communication (PLC) is a communication technology that uses a power line as a communication medium. PLC technology requires a transmitter, a receiver and a communication medium. The communication medium of PLC is a power line. The transmitter modulates and then couples the signal into the power line where it travels to the receiver at the opposing end of the communication link which demodulates the signals. The power line itself may be AC or DC. The communication between a transmitter and a receiver may be bidirectional. In the bidirectional case, the devices will transmit and receive, thus they may be characterized as transceivers.

PLC implements modulation schemes to couple the data as a frequency to be transmitted onto the AC or DC power line. A variety of modulation schemes exist for PLC, including Amplitude Shift Keying (ASK), Frequency Shift Keying (FSK) including Binary Frequency Shift Keying (BFSK), and Phase Shift Keying (PSK) including Binary Phase Shift Keying (BPSK). In one preferred embodiment, BPSK is used to communicate over the power conductors in the driveline, thereby eliminating the need for separate conductors in the driveline for transmit/receive, which can reduce the diameter of the driveline and thereby reduce the likelihood of driveline infection at the site where the driveline passes through a patient's skin. Of the keying schemes listed above, BPSK is suitable because BPSK is resistant to failures due to a conductor's reduced impedance over time due to corrosion of contacts or wires in the patient's body. Note, however, that ASK, FSK, BFSK and PSK could also be used in the driveline disclosed herein.

Referring to FIG. 8, a suitable BPSK Modulator 810 is shown that includes a bipolar non-return-to-zero level encoder 820 that receives a signal input, a carrier signal generator 840, and a mixer circuit 830. The bipolar non-return-to-zero level encoder 820 converts the signal input to be modulated into an equivalent bipolar non-return-to-zero sequence. This bipolar non-return-to-zero sequence is fed into the mixer circuit 830 together with the carrier signal output from the carrier signal generator 840 to form the final BPSK modulated signal on the signal output.

The BPSK modulator 810 is used at the transmit end to encode a digital message on the signal output. The receive end will have a BPSK demodulator, one example of which is shown as 910 in FIG. 9. The BPSK demodulator 910 receives a signal input, which is the signal output from the BPSK modulator 810 in FIG. 8. Coherent detection is used to demodulate the BPSK signal input. Using the coherent detection technique, the knowledge of the carrier frequency and phase must be known to the receiver. This can be achieved by using a Phase Lock Loop (PLL) 930. Hence, as shown in FIG. 9, the BPSK demodulator 910 includes a PLL 930, a multiplier circuit 920, an integrator 940, a bit synchronizer 950, a switch circuit 960 and a comparator 970. The received signal at the signal input is multiplied by a reference frequency signal from the PLL 930 via the multiplier circuit 920. The multiplied output is integrated over a one-bit period using the integrator 940, bit synchronizer 950 and the switch circuit 960. The comparator 970 makes a decision on each integrated bit based on a threshold. Because non-return-to-zero signaling format was used in the BPSK modulator 810, the threshold for the comparator 970 is set to zero. The equivalent binary data generated after the comparison is the demodulated signal output. The BPSK technique provides better noise immunity and utilizes less bandwidth compared to BFSK.

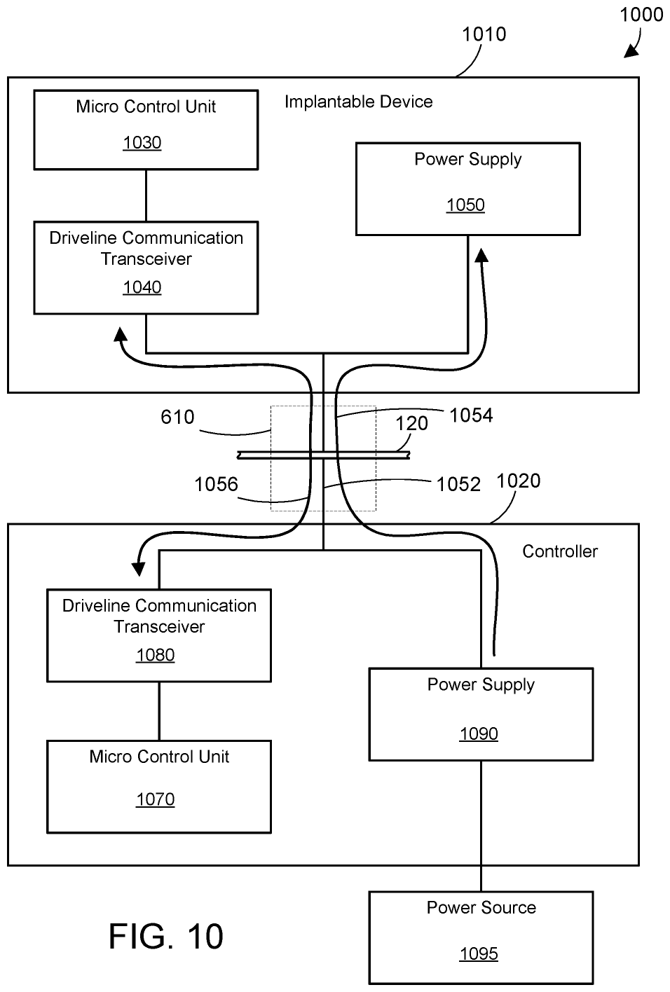

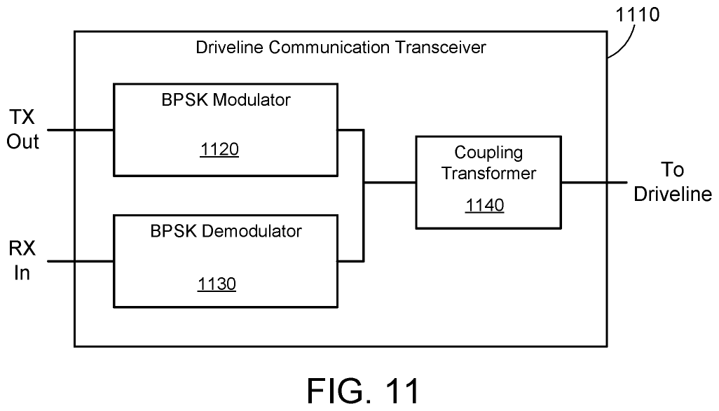

FIG. 10 shows a block diagram of a system 1000, which is one suitable implementation that includes features of system 700 shown in FIG. 7. System 1000 preferably includes an implantable device 1010 that includes a micro control unit 1030, a driveline communication transceiver 1040, and a power supply 1050. The micro control unit 1030 preferably includes a microcontroller running code to control suitable mechanical devices that perform the desired function of the implantable device 1010, such as pumping blood. The driveline communication transceiver 1040 preferably includes a BPSK modulator and demodulator as discussed above with reference to transceivers 720 and 740 in FIG. 7. The power supply 1050 preferably can switch between an internal battery and a source of DC power provided via the driveline 610 from the controller 1020. The internal battery in the power supply 1050 is provided to assure the implantable device 1010 continues to function should the controller 1020 be disconnected for a short period of time to allow replacing the controller 1020 with a different controller. There are some implantable devices that do not have internal power supplies. For implantable devices that do not have an internal power supply, the power supply 1050 in FIG. 10 could be omitted. The disclosure and claims herein expressly extend to a driveline system for all active implantable devices, whether or not they have an internal power supply.

The controller 1020 preferably includes a micro control unit 1070, a driveline communication transceiver 1080, and a power supply 1090. The micro control unit 1070 preferably includes a microcontroller running code that controls the function of the implantable device 1010 by sending digital messages to the implantable device 1010. The driveline communication transceiver 1080 receives a digital message from the micro control unit 1070, encodes the message via a BPSK modulator, and transmits the message on the driveline 610 to the driveline communication transceiver 1040 in the implantable device 1010. The driveline communication transceiver 1040 decodes the digital message and sends the digital message to the micro control unit 1030, which then performs a desired function corresponding to the received message.

The power supply 1090 provides power to the controller 1020 and to the implantable device 1010 via the driveline 610. The power supply 1090 preferably includes a rechargeable battery capable of powering the controller and implantable device for several hours. The power supply 1090 may be coupled to an external power source, such as an AC power outlet, to provide power to the controller 1020 and implantable device 1010 and to recharge the battery internal to the power supply 1090. In one suitable implementation, the power supply 1090 in the controller is a rechargeable battery that can be connected to an external power supply that includes its own rechargeable battery and an AC power source. The controller 1020 can then choose a power source from its own battery, the battery in the external power source, or the power from the AC power source, depending on whether or not the controller is plugged into the external power source and the power levels of the rechargeable batteries in the controller and power source, or other factors.

The driveline 610 connects the controller 1020 to the implantable device 1010. The single line 1052 shown in FIG. 9 includes the four conductors 620, 630, 640 and 650 shown in FIGS. 6 and 7. The driveline 610 provides redundant positive polarity power connections over conductors 620 and 640 and provides redundant negative polarity power connections over conductors 630 and 650, as indicated by arrow 1054. These same four conductors in the driveline 610 also provide bidirectional digital communication via BPSK between the two driveline communication transceivers 1040 and 1080, as indicated by arrow 1056 in FIG. 10. The driveline system as disclosed herein provides a driveline with four conductors that provide redundant power and redundant communication capability over the same four conductors.

Referring to FIG. 11, a driveline communication transceiver 1110 is one suitable implementation for the driveline communication transceivers 720 and 740 in FIG. 7 and 1040 and 1080 in FIG. 10. The driveline communication transceiver 1110 includes a BPSK modulator 1120, a BPSK demodulator 1130, and a coupling transformer 1140. The driveline communication transceiver 1110 provides a transmission path from the TX Out input through the BPSK modulator 1120 and coupling transformer 1140 to the driveline. The driveline communication transceiver 1110 also provides a receive path from the driveline through the coupling transformer 1140 and BPSK demodulator 1130 to the RX In output.

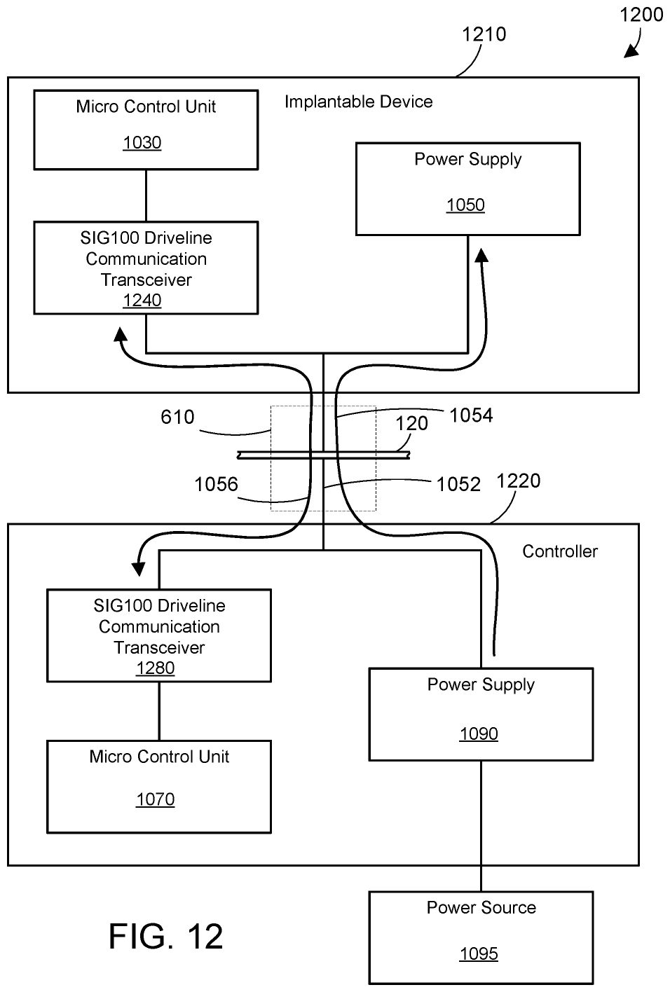

In one suitable implementation, the driveline communication transceivers are SIG100 UART/LIN over Powerline Transceivers from Yamar company. This implementation is shown as system 1200 in FIG. 12. The primary difference between the system 1200 in FIG. 12 and the system 1000 in FIG. 10 is the two driveline communication transceivers 1040 and 1080 in FIG. 10 have been replaced with the SIG100 driveline communication transceivers 1240 and 1280 in the implantable device 1210 and controller 1220, respectively, in FIG. 12.

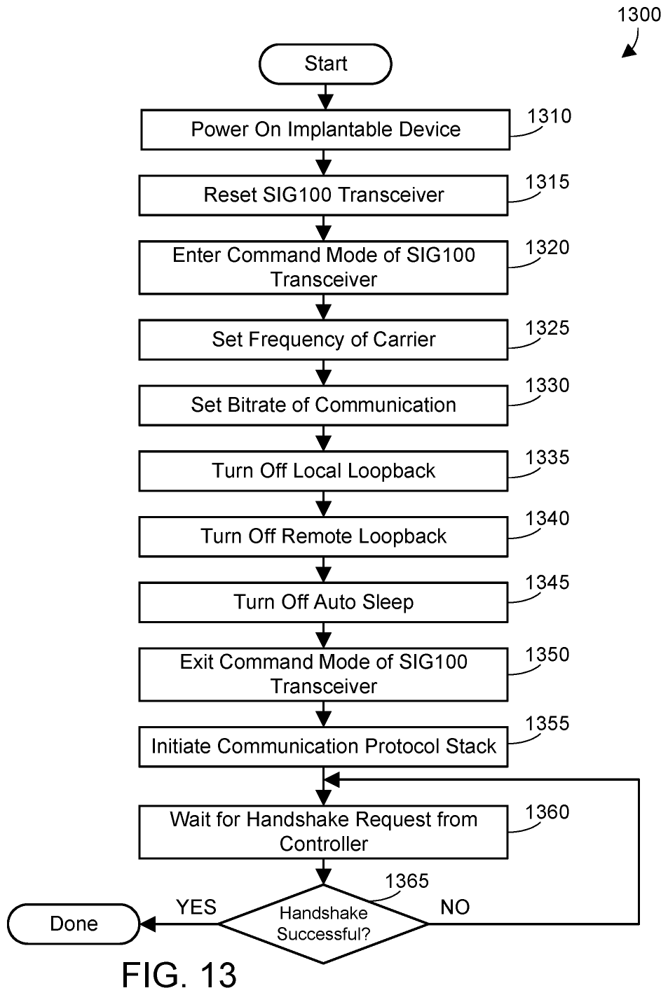

Referring to FIG. 13, a method 1300 is used to setup the SIG100 driveline communication transceiver 1240 in the implantable device 1210 shown in FIG. 12. Power on the implantable device (step 1310). Reset the SIG100 transceiver (step 1315). Enter the command mode of the SIG100 transceiver (step 1320). Set the frequency of the carrier (step 1325). Set the bitrate of communication (step 1330). Turn off local loopback (step 1335). Turn off remote loopback (step 1340). Turn off auto sleep (step 1345). Exit the command mode of the SIG100 transceiver (step 1350). Initiate the communication protocol stack (step 1355). Wait for a handshake request from the controller (step 1360). If the handshake is not successful (step 1365=NO), method 1300 loops back to step 1360 and awaits another handshake request from the controller (step 1360). When the handshake is successful (step 1365=YES), method 1300 is done, and the communication transceiver 1240 in the implantable device 1210 is ready for communications to and from the controller.

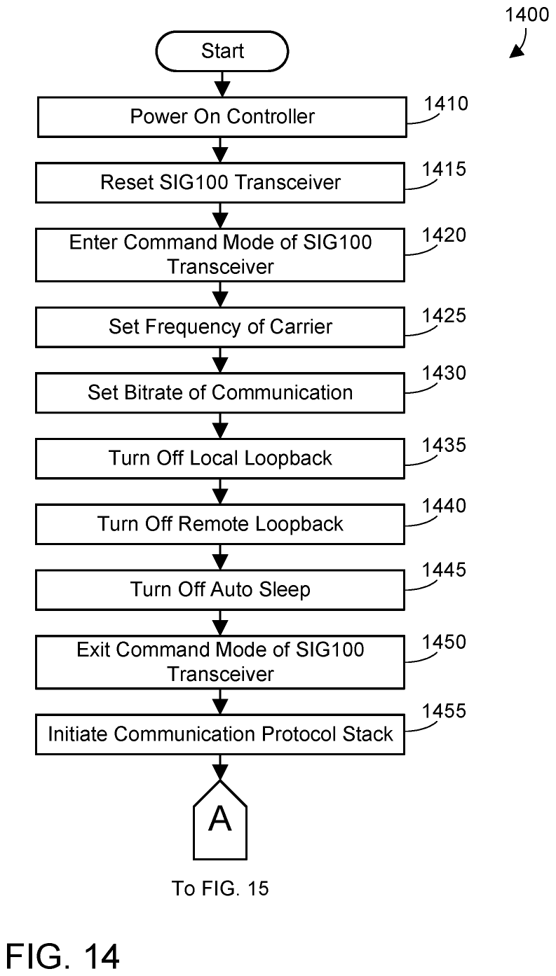

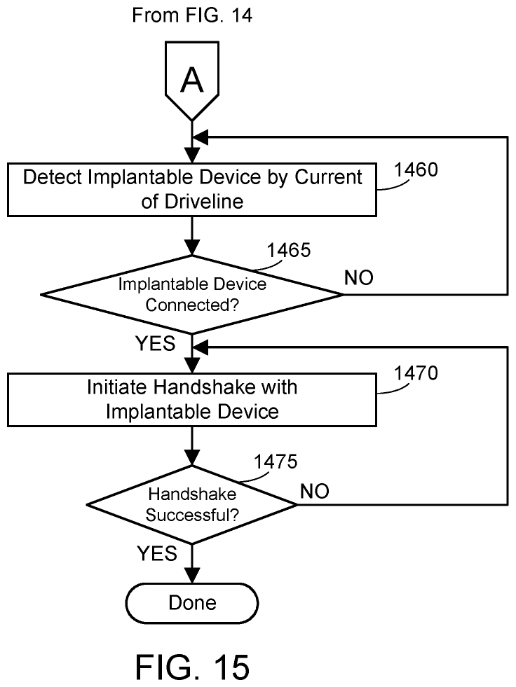

Referring to FIGS. 14 and 15, a method 1400 is used to setup the SIG100 driveline communication transceiver 1280 in the controller 1220 shown in FIG. 12. Power on the controller (step 1410). Reset the SIG100 transceiver (step 1415). Enter the command mode of the SIG100 transceiver (step 1420). Set the frequency of the carrier (step 1425). The frequency of the carrier in step 1425 is set to match the frequency of the carrier in step 1325 in FIG. 13. Set the bitrate of communication (step 1430). The bitrate of communication in step 1430 is set to match the bitrate of communication in step 1330 in FIG. 13. Turn off local loopback (step 1435). Turn off remote loopback (step 1440). Turn off auto sleep (step 1445). Exit the command mode of the SIG100 transceiver (step 1450). Initiate the communication protocol stack (step 1455). Detect an implantable device by sensing current in the driveline (step 1460). As long as the implantable device is not connected (step 1465=NO), method 1400 loops back to step 1460 and waits. Once the implantable device is connected (step 1465=YES), the controller initiates a handshake with the implantable device (step 1470). If the handshake is not successful (step 1475=NO), method 1400 loops back to step 1470 and initiates a handshake again. Once the handshake is successful (step 1475=YES), method 1400 is done, which means the driveline system is now ready to communicate over the power line connections between the controller and the implantable device.

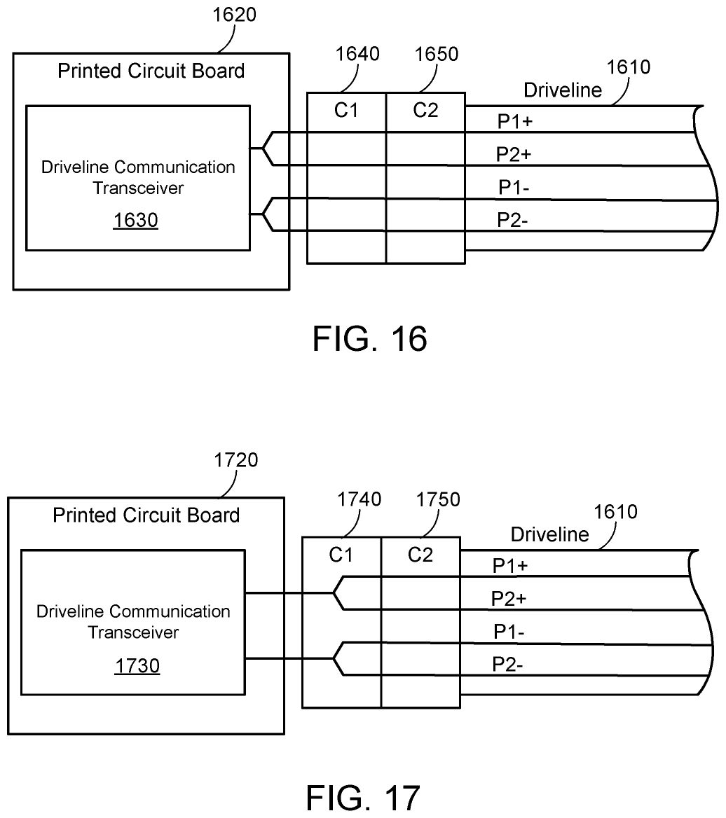

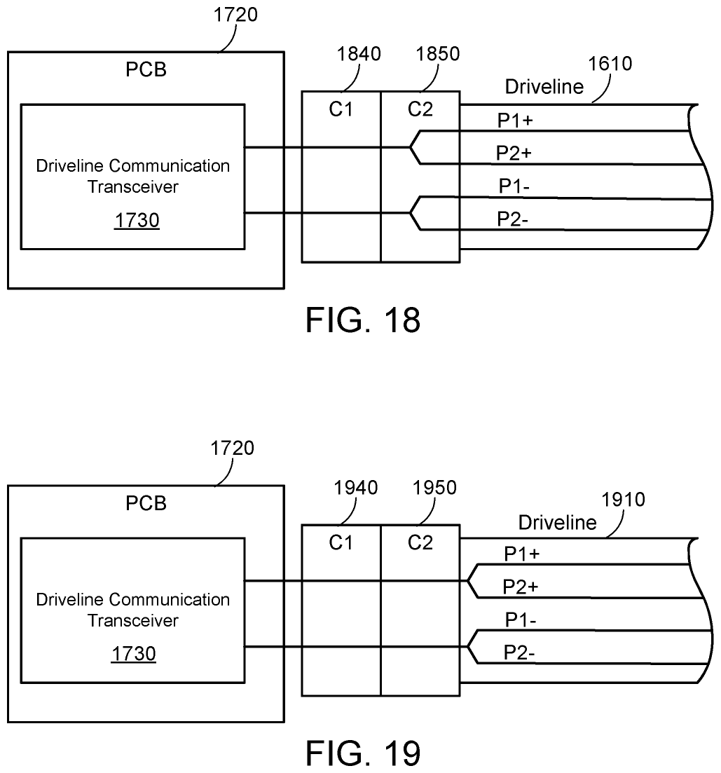

The interconnection between conductors 620 and 640 and the interconnection between conductors 630 and 650 in FIGS. 6 and 7 can be made in any suitable location and in any suitable way. FIGS. 16-19 show different locations for making the interconnection between conductors in the driveline. Referring to FIG. 16, a driveline communication transceiver 1630 is connected to a printed circuit board 1620, and connections on the printed circuit board interconnect the two pairs of conductors as shown in FIG. 16 so both pairs of conductors are connected to the driveline 1610 through connectors 1640 and 1650 as shown. Note the printed circuit board 1620 and driveline communication transceiver 1630 could be in either the controller or the implantable device. Referring to FIG. 17, the pairs of conductors could be interconnected within a connector 1740, which mates to a suitable mating connector 1750 on the driveline 1610. Referring to FIG. 18, the pairs of conductors could be interconnected within a connector 1850 on the driveline 1610, which mates to a suitable mating connector 1840 to connect to the driveline communication transceiver 1730. Referring to FIG. 19, the pairs of conductors could be interconnected within the driveline 1910 itself, which includes a connector 1950 with two conductors that mates to a corresponding mating connector 1940 that is connected to the driveline communication transceiver 1730. The printed circuit board and driveline communication transceiver in FIGS. 16-19 could be in either the controller or the implantable device. The driveline 1610 in FIGS. 16-18 and the driveline 1910 in FIG. 19 are both specific implementations of the driveline 610 shown in FIGS. 6 and 7.

While the specific examples in FIGS. 16-19 show interconnecting the pairs of conductors in the driveline in the same locations (printed circuit board 1620 in FIG. 16, connector C1 1740 in FIG. 17, connector C2 1850 in FIG. 18 and driveline 1910 in FIG. 19), the interconnections could occur in different locations. Thus, the conductors corresponding to P1+ and P2+ could be interconnected on the printed circuit board 1620 in FIG. 16 while the conductors corresponding to P1− and P2− could be interconnected in the connector C2 1850 shown in FIG. 18. The disclosure and claims herein expressly extend to any suitable location or combination of locations for interconnecting the pairs of conductors in the driveline.

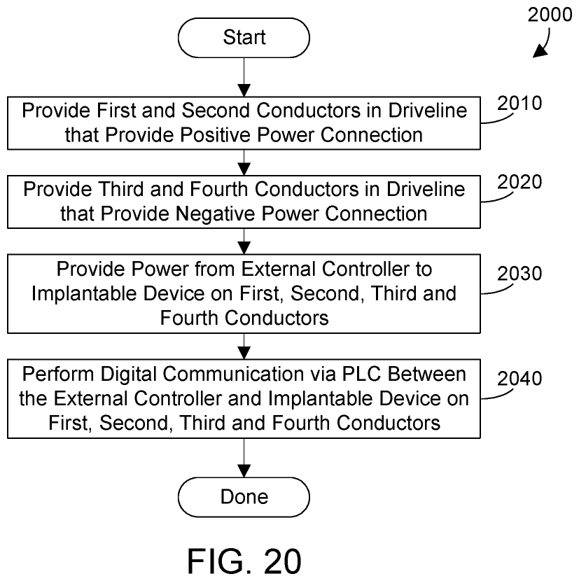

Referring to FIG. 20, a method 2000 is performed in accordance with the preferred embodiment. Provide first and second conductors in the driveline that provide a positive power connection (step 2010). Provide third and fourth conductors in the driveline that provide a negative power connection (step 2020). Provide power from the external controller to the implantable device on the first, second, third and fourth conductors (step 2030). Perform digital communication via PLC between the external controller and the implantable device on the first, second, third and fourth conductors (step 2040). Method 2000 is then done.

In FIG. 20, the terms “positive power connection” and “negative power connection” are used. These terms are used as specific examples of power in a first polarity and power in a second polarity, which are intended in their broadest possible scope to include any suitable connections that can provide power from the controller to the implantable device, including without limitation both AC and DC connections. In the most preferred implementation, because the driveline system involves an implantable device that is implanted into a patient's body, a DC connection is preferred. For example, the power of a first polarity could be a suitable DC voltage, such as +3 volts DC, while the power in the second polarity could be a ground connection or a connection to a suitable DC voltage that is higher than, but preferably lower than, the power of the first polarity.

A driveline and driveline system for an active implantable device provides for communication over the power conductors in the driveline. The driveline includes four conductors, two for the positive power connection and two for the negative power connection. Communication in the driveline is performed using Power Line Communication (PLC) technology on the four conductors, superimposing a communication signal on the conductors such that the four conductors provide both power and communication capability. The four conductors provide redundant paths for both power and communication, so the failure of one of the conductors in the driveline does not affect operation of the driveline. The four conductor driveline cable and system provides a more robust driveline cable that does not fail with the failure of one of the conductors without increasing the diameter of the driveline, which would increase the risk of infection where the driveline passes through the skin.

The disclosure and claims herein support a driveline for an implantable medical device that connects the implantable medical device to an external controller, wherein the driveline comprises: first and second power conductors that provide a first polarity of power from the external controller to the implantable medical device, wherein the first and second power conductors are interconnected; and third and fourth power conductors that provide a second polarity of power from the external controller to the implantable medical device, wherein the third and fourth power conductors are interconnected.

The disclosure and claims herein further support a driveline system for an implantable medical device that connects the implantable medical device to an external controller, wherein the driveline system comprises: (A) a driveline comprising: first and second power conductors that provide a first polarity of power from the external controller to the implantable medical device, wherein the first and second power conductors are interconnected; third and fourth power conductors that provide a second polarity of power from the external controller to the implantable medical device, wherein the third and fourth power conductors are interconnected; (B) a first driveline communication transceiver in the implantable medical device and coupled to the first, second, third and fourth power conductors in the driveline that provides digital communication to and from the external controller over the first, second, third and fourth power conductors in the driveline; and (C) a second driveline communication transceiver in the external controller and coupled to the first, second, third and fourth power conductors in the driveline that provides digital communication to and from the implantable device over the first, second, third and fourth power conductors in the driveline.

The disclosure and claims herein additionally support a method for an external controller to communicate with an implantable device on a driveline that connects the external controller to the implantable device, the method comprising: providing first and second conductors in the driveline that provide a positive power connection from the external controller to the implantable device; providing third and fourth conductors in the driveline that provide a negative power connection from the external controller to the implantable device; providing power from the external controller to the implantable device on the first, second, third and fourth conductors; and performing digital communication between the external controller and the implantable device on the first, second, third and fourth conductors.

While the invention has been particularly shown and described with reference to preferred embodiments thereof, it will be understood by those skilled in the art that various changes in form and details may be made therein without departing from the spirit and scope of the invention.

1. A driveline for an implantable medical device that connects the implantable medical device to an external controller, wherein the driveline comprises:
first and second power conductors that provide a first polarity of power from the external controller to the implantable medical device, wherein the first and second power conductors are interconnected; and
third and fourth power conductors that provide a second polarity of power from the external controller to the implantable medical device, wherein the third and fourth power conductors are interconnected.

2. The driveline of claim 1 wherein the first and third power conductors are connected in the implantable medical device.

3. The driveline of claim 1 wherein the first and third power conductors are connected in a connector to the driveline.

4. The driveline of claim 1 wherein the first and third power conductors are connected in the driveline.

5. The driveline of claim 1 wherein the first and third power conductors are connected in the external controller.

6. The driveline of claim 1 wherein the second and fourth power conductors are connected in the implantable medical device.

7. The driveline of claim 1 wherein the second and fourth power conductors are connected in a connector to the driveline.

8. The driveline of claim 1 wherein the second and fourth power conductors are connected in the driveline.

9. The driveline of claim 1 wherein the second and fourth power conductors are connected in the external controller.

10. A driveline system for an implantable medical device that connects the implantable medical device to an external controller, wherein the driveline system comprises:
(A) a driveline comprising:
first and second power conductors that provide a first polarity of power from the external controller to the implantable medical device, wherein the first and second power conductors are interconnected;
third and fourth power conductors that provide a second polarity of power from the external controller to the implantable medical device, wherein the third and fourth power conductors are interconnected;
(B) a first driveline communication transceiver in the implantable medical device and coupled to the first, second, third and fourth power conductors in the driveline that provides digital communication to and from the external controller over the first, second, third and fourth power conductors in the driveline; and
(C) a second driveline communication transceiver in the external controller and coupled to the first, second, third and fourth power conductors in the driveline that provides digital communication to and from the implantable device over the first, second, third and fourth power conductors in the driveline.

11. The driveline system of claim 10 wherein the first and third power conductors are connected in the implantable medical device.

12. The driveline system of claim 10 wherein the first and third power conductors are connected in a connector to the driveline.

13. The driveline system of claim 10 wherein the first and third power conductors are connected in the driveline.

14. The driveline system of claim 10 wherein the first and third power conductors are connected in the external controller.

15. The driveline system of claim 10 wherein the second and fourth power conductors are connected in the implantable medical device.

16. The driveline system of claim 10 wherein the second and fourth power conductors are connected in a connector to the driveline.

17. The driveline system of claim 10 wherein the second and fourth power conductors are connected in the driveline.

18. The driveline system of claim 10 wherein the second and fourth power conductors are connected in the external controller.

19. The driveline system of claim 10 wherein the first and second driveline communication transceivers communicate with each other using binary phase shift keying (BPSK) modulation and demodulation.

20. The driveline system of claim 10 wherein the first and second driveline communication transceivers each comprise:
a coupling transformer coupled to at least one conductor in the driveline;
a binary phase shift keying (BPSK) modulator coupled to the coupling transformer that drives a transmitted message via the coupling transformer to the driveline; and
a BPSK demodulator coupled to the coupling transformer that receives a message on the driveline received via the coupling transformer.

21. The driveline system of claim 10 wherein the driveline system maintains power from the external controller to the implantable device and further maintains communication between the external controller and the implantable device when one of the first and second power conductors fails.

22. The driveline system of claim 10 wherein the driveline system maintains power from the external controller to the implantable device and further maintains communication between the external controller and the implantable device when one of the third and fourth power conductors fails.

23. The driveline system of claim 10 wherein the driveline system maintains power from the external controller to the implantable device and further maintains communication between the external controller and the implantable device when one of the first and second power conductor fails at the same time when one of the third and fourth power conductors has failed.

24. A method for an external controller to communicate with an implantable device on a driveline that connects the external controller to the implantable device, the method comprising:
providing first and second conductors in the driveline that provide a positive power connection from the external controller to the implantable device;
providing third and fourth conductors in the driveline that provide a negative power connection from the external controller to the implantable device;
providing power from the external controller to the implantable device on the first, second, third and fourth conductors; and
performing digital communication between the external controller and the implantable device on the first, second, third and fourth conductors.

25. The method of claim 24 wherein the implantable medical device comprises a first driveline communication transceiver and the external controller comprises a second driveline communication transceiver, the method further comprising:
the first and second driveline communication transceivers communicating with each other over the driveline using binary phase shift keying (BPSK) modulation and demodulation.
